SEQUENCE LISTING (79 sequences)

SEQ ID NO: 1 - DNA, 80 bp, Artificial Sequence
  Synthetic Selection Library
cgctctcgtccatgtgttggnnnnnnnnnnnnnnnnnnnnnnnnnnnnnnnnnnnnnnnnccacacgatgcgtagttccg

SEQ ID NO: 2 - DNA, 20 bp, Artificial Sequence
  Synthetic Forward primer
cgctctcgtccatgtgttgg

SEQ ID NO: 3 - DNA, 20 bp, Artificial Sequence
  Synthetic Primer
cggaactacgcatcgtgtgg

SEQ ID NO: 4 - DNA, 80 bp, Artificial Sequence
  Synthetic DNA ligand (Afla-17-2)
cgctctcgtccatgtgttgggcacgtgttgtctctctgtgtctcgtgcccttcgctaggcccacacgatgcgtagttccg

SEQ ID NO: 5 - DNA, 80 bp, Artificial Sequence
  Synthetic DNA ligand (Afla-17-5)
cgctctcgtccatgtgttggctgcgtcccttcgtcgtctccctgtgctcggaagggattgccacacgatgcgtagttccg

SEQ ID NO: 6 - DNA, 80 bp, Artificial Sequence
  Synthetic DNA ligand (Afla-17-7)
cgctctcgtccatgtgttgggcagctaaaattataaagtaattctatgctggtttaggggccacacgatgcgtagttccg

SEQ ID NO: 7 - DNA, 80 bp, Artificial Sequence
  Synthetic DNA ligand (Afla-17-8)
cgctctcgtccatgtgttggcaatgtcggcatggccatctataaagtagatggtgtgcccccacacgatgcgtagttccg

SEQ ID NO: 8 - DNA, 80 bp, Artificial Sequence
  Synthetic DNA ligand (Afla-17-9)
cgctctcgtccatgtgttgggcggatagcaggtaacggatccgctatcctatcgccacagccacacgatgcgtagttccg

SEQ ID NO: 9 - DNA, 80 bp, Artificial Sequence
  Synthetic DNA ligand (Afla-17-10)
cgctctcgtccatgtgttggcgtgacgcccgtcgtatgtactttatacctagacgtgcgcccacacgatgcgtagttccg

SEQ ID NO: 10 - DNA, 80 bp, Artificial Sequence
  Synthetic DNA ligand (Afla-17-19)
cgctctcgtccatgtgttggcgtgacgcccgtcgtatgtactttatacctagacgtgcgaccacacgatgcgtagttccg

SEQ ID NO: 11 - DNA, 80 bp, Artificial Sequence
  Synthetic DNA ligand (Afla-17-18)
cgctctcgtccatgtgttgggaaccccataattcactgtataaagtactgtgaatcaccgccacacgatgcgtagttccg

SEQ ID NO: 12 - DNA, 80 bp, Artificial Sequence; identical to SEQ ID NO: 4
  Synthetic DNA ligand (Afla-17-11)

SEQ ID NO: 13 - DNA, 80 bp, Artificial Sequence
  Synthetic DNA ligand (Afla-17-12)
cgctctcgtccatgtgttgggagtagctatacaaacgtatcactttatgctagtttgtcgccacacgatgcgatgttccg

SEQ ID NO: 14 - DNA, 80 bp, Artificial Sequence
  Synthetic DNA ligand (Afla-17-15)
cgctctcgtccatgtgttggcagggaggaggaattataaagtaattcctaatgtgcagtaccacacgatgcgtagttccg

SEQ ID NO: 15 - DNA, 80 bp, Artificial Sequence
  Synthetic DNA ligand (Afla-17-3)
cgctctcgtccatgtgttgggcaggattgagtataaagtactaaatctatccgacctgtgccacacgatgcgtagttccg

SEQ ID NO: 16 - DNA, 80 bp, Artificial Sequence
  Synthetic DNA ligand (Afla-17-6)
cgctctcgtccatgtgttgggggcgccgtatcgtactttatacgctaggccttcgtttgcccacacgatgcgtagttccg

SEQ ID NO: 17 - DNA, 61 bp, Artificial Sequence
  Synthetic DNA ligand (OTA1.4)
tggtggctgtaggtcagcacgatggggaaagggtccccctgggttggagcatcggacaacg

SEQ ID NO: 18 - DNA, 58 bp, Artificial Sequence
  Synthetic DNA ligand (Z1.4.2)
tggtggctgtaggtcagcacgatggggaaagggtccccctgggttggagcatcggaca

SEQ ID NO: 19 - DNA, 38 bp, Artificial Sequence
  Synthetic DNA ligand (Z1.4.3)
gatggggaaagggtccccctgggttggagcatcggaca

SEQ ID NO: 20 - DNA, 36 bp, Artificial Sequence
  Synthetic DNA ligand (Z1.4.4)
ggctgtaggtcagcacgatggggaaagggtccccct

SEQ ID NO: 21 - DNA, 80 bp, Artificial Sequence
  Synthetic DNA ligand (Afla-17-1)
cgctctcgtccatgtgttgggcttgttatggctcatgttgtctccctgtgttatgggccaccacacgatgcgtagttccg

SEQ ID NO: 22 - DNA, 80 bp, Artificial Sequence
  Synthetic DNA ligand (Afla-17-4)
cgctctcgtccatgtgttggccaacacgagtcgaactataaagtagaacgacatataccaccacacgatgcgtagttccg

SEQ ID NO: 23 - DNA, 80 bp, Artificial Sequence
  Synthetic DNA ligand (Afla-17-14)
cgctctcgtccatgtgttgggtcgcgacctgttctctctgtgcgctaggtcgccagctgtccacacgatgcgtagttccg

SEQ ID NO: 24 - DNA, 80 bp, Artificial Sequence
  Synthetic DNA ligand (Afla-17-23)
cgctctcgtccatgtgttggccatgggaaggcgtatattctttacaatctagccgaccagccacacgatgcgtagttccg

SEQ ID NO: 25 - DNA, 79 bp, Artificial Sequence
  Synthetic DNA ligand (Afla-17-25)
cgctctcgtccatgtgttggaaaaggcgtttataaagtaaacggtttagaatgtctgcaccacacgatgcgtagttccg

SEQ ID NO: 26 - DNA, 80 bp, Artificial Sequence
  Synthetic DNA ligand (Afla-17-27)
cgctctcgtccatgtgttggtggatcccgagcattagtttactttatagtttaatccgggccacacgatgcgtagttccg

SEQ ID NO: 27 - DNA, 80 bp, Artificial Sequence
  Synthetic DNA ligand (Afla-17-29)
cgctctcgtccatgtgttggagcgggcgtataatttctttaaaattctagcactccacagccacacgatgcgtagttccg

SEQ ID NO: 28 - DNA, 80 bp, Artificial Sequence
  Synthetic DNA ligand (Afla-17-30)
cgctctcgtccatgtgttggggctgctcgactgttgtctctctgtgtttgtcgtgccctgccacacgatgcgtagttccg

SEQ ID NO: 29 - DNA, 80 bp, Artificial Sequence
  Synthetic DNA ligand (Afla-17-31)
cgctctcgtccatgtgttggaccaaaatatcaaagcacagagagacaacttgagcactgaccacacgatgcgtagttccg

SEQ ID NO: 30 - DNA, 80 bp, Artificial Sequence
  Synthetic DNA ligand (Afla-17-33)
cgctctcgtccatgtgttgggtgtggagacgctcatgctataaagtagcatgatctaggcccacacgatgcgtagttccg

SEQ ID NO: 31 - DNA, 80 bp, Artificial Sequence
  Synthetic DNA ligand (Afla-17-34)
cgctctcgtccatgtgttgggaggcgcaagagaaccataaagtgaacctcatagcacagaccacacgatgcgtagttccg

SEQ ID NO: 32 - DNA, 80 bp, Artificial Sequence
  Synthetic DNA ligand (Afla-17-35)
cgctctcgtccatgtgttggacaacttcaagctagtttactttatagtttagctgggtggccacacgatgcgtagttccg

SEQ ID NO: 33 - DNA, 80 bp, Artificial Sequence
  Synthetic DNA ligand (Afla-17-36)
cgctctcgtccatgtgttggtcaggggaggactataaagtagtgctccattcttcgggggccacacgatgcgtagttccg

SEQ ID NO: 34 - DNA, 80 bp, Artificial Sequence
  Synthetic DNA ligand (Afla-17-37)
cgctctcgtccatgtgttggacgggcgaggggatcctctggtggtgtccttgccctttggccacacgatgcgtagttccg

SEQ ID NO: 35 - DNA, 80 bp, Artificial Sequence
  Synthetic DNA ligand (Afla-17-39)
cgctctcgtccatgtgttggagcgggaggtgtgttgtctctctgtgtttcccctccctcaccacacgatgcgtagttccg

SEQ ID NO: 36 - DNA, 80 bp, Artificial Sequence
  Synthetic DNA ligand (Afla-17-40)
cgctctcgtccatgtgttggagccacgaaagcgtacataactttattatttagctatgccccacacgatgcgtagttccg

SEQ ID NO: 37 - DNA, 22 bp, Artificial Sequence
  Synthetic DNA ligand (Afla-17-6-1)
cgtatcgtactttatacgctag

SEQ ID NO: 38 - DNA, 28 bp, Artificial Sequence
  Synthetic DNA ligand (Afla-17-6-2)
cgccgtatcgtactttatacgctaggcc

SEQ ID NO: 39 - DNA, 29 bp, Artificial Sequence
  Synthetic DNA ligand (Afla-17-6-3)
cgccgtatcgtactttatacgctaggcct

SEQ ID NO: 40 - DNA, 32 bp, Artificial Sequence
  Synthetic DNA ligand (Afla-17-6-4)
cgccgtatcgtactttatacgctaggccttcg

SEQ ID NO: 41 - DNA, 40 bp, Artificial Sequence
  Synthetic DNA ligand (Afla-17-6-5)
gggcgccgtatcgtactttatacgctaggccttcgtttgc

SEQ ID NO: 42 - DNA, 49 bp, Artificial Sequence
  Synthetic DNA ligand (Afla-17-6-2-1)
ttgggggcgccgtatcgtactttatacgctaggccttcgtttgccccac

SEQ ID NO: 43 - DNA, 53 bp, Artificial Sequence
  Synthetic DNA ligand (Afla-17-6-2-2)
tgttgggggcgccgtatcgtactttatacgctaggccttcgtttgccccacac

SEQ ID NO: 44 - DNA, 48 bp, Artificial Sequence
  Synthetic DNA ligand (Afla-17-6-2-3)
ttgggggcgccgtatcgtactttatacgctaggccttcgtttgcccac

SEQ ID NO: 45 - DNA, 70 bp, Artificial Sequence
  Synthetic DNA ligand (Afla-17-2-1)
tcgtccatgtgttgggcacgtgttgtctctctgtgtctcgtgcccttcgctaggcccacaccatgcgtag

SEQ ID NO: 46 - DNA, 60 bp, Artificial Sequence
  Synthetic DNA ligand (Afla-17-2-2)
catgtgttgggcacgtgttgtctctctgtgtctcgtgcccttcgctaggcccacacgatg

SEQ ID NO: 47 - DNA, 50 bp, Artificial Sequence
  Synthetic DNA ligand (Afla-17-2-3)
gttgggcacgtgttgtctctctgtgtctcgtgcccttcgctaggcccaca

SEQ ID NO: 48 - DNA, 31 bp, Artificial Sequence
  Synthetic DNA ligand (Afla-17-2-4)
atgtgttgggcacgtgtcgtgcccttcgcta

SEQ ID NO: 49 - DNA, 34 bp, Artificial Sequence
  Synthetic DNA ligand (Afla-17-2-5)
tgggcacgtgttgtctctctgtgtctcgtgccct

SEQ ID NO: 50 - DNA, 40 bp, Artificial Sequence
  Synthetic DNA ligand (Afla-17-2-6)
gcacgtgttgtctctctgtgtctcgtgcccttcgctaggc

SEQ ID NO: 51 - DNA, 19 bp, Artificial Sequence
  Synthetic DNA ligand (Afla-17-2-7)
tgggcacgtgtcgtgccct

SEQ ID NO: 52 - DNA, 80 bp, Artificial Sequence
  Synthetic DNA ligand (Afla-21-1)
cgctctcgtccatgtgttgggggtagcgtgcttaggcataaagagcatacagacacggcgccacacgatgcgtagttccg

SEQ ID NO: 53 - DNA, 80 bp, Artificial Sequence
  Synthetic DNA ligand (Afla-21-2)
cgctctcgtccatgtgttggaacaacaaaacgtggacctataaagtaggaccaagtcggcccacacgatgcgtagttccg

SEQ ID NO: 54 - DNA, 80 bp, Artificial Sequence
  Synthetic DNA ligand (Afla-21-3)
cgctctcgtccatgtgttggctaacacgggcgatgtataaagtacaatcggcgacagggcccacacgatgcgtagttccg

SEQ ID NO: 55 - DNA, 80 bp, Artificial Sequence
  Synthetic DNA ligand (Afla-21-5)
cgctctcgtccatgtgttggtcaacgtcgtacaattcttcacaattctagacgtccccggccacacgatgcgtagttccg

SEQ ID NO: 56 - DNA, 80 bp, Artificial Sequence
  Synthetic DNA ligand (Afla-21-7)
cgctctcgtccatgtgttggaggataaactagattgatagagacaatgtacgtttcgaggccacacgatgcgtagttccg

SEQ ID NO: 57 - DNA, 80 bp, Artificial Sequence
  Synthetic DNA ligand (Afla-21-8)
cgctctcgtccatgtgttggcggcatcgcatggggtcataaagtgcccgtccattctccaccacacgatgcgtagttccg

SEQ ID NO: 58 - DNA, 80 bp, Artificial Sequence
  Synthetic DNA ligand (Afla-21-9)
cgctctcgtccatgtgttggcatgcgtatatgactttatcatctagctccggacctcgtcccacacgatgcgtagttccg

SEQ ID NO: 59 - DNA, 80 bp, Artificial Sequence
  Synthetic DNA ligand (Afla-21-10)
cgctctcgtccatgtgttggttgatccctctaatcgtcctataaagtaggacgtaccgccccacacgatgcgtagttccg

SEQ ID NO: 60 - DNA, 80 bp, Artificial Sequence; identical to SEQ ID NO: 34
  Synthetic DNA ligand (Afla-21-12)

SEQ ID NO: 61 - DNA, 80 bp, Artificial Sequence
  Synthetic DNA ligand (Afla-21-13)
cgctctcgtccatgtgttggcccccaaaaccgtaccatccttgcatgctaggtttttccaccacacgatgcgtagttccg

SEQ ID NO: 62 - DNA, 79 bp, Artificial Sequence
  Synthetic DNA ligand (Afla-21-14)
cgctctcgtccatgtgttgggtgcgtacgtgtactttatacacctagcttcagatcggcccacacgatgcgtagttccg

SEQ ID NO: 63 - DNA, 81 bp, Artificial Sequence
  Synthetic DNA ligand (Afla-21-15)
cgctctcgtccatgtgttggtggaggggtcacccgtacaagctttacttctagggtgacggccacacgatgcgtagttccg

SEQ ID NO: 64 - DNA, 80 bp, Artificial Sequence
  Synthetic DNA ligand (Afla-21-16)
cgctctcgtccatgtgttggtaagcggggtgtacgtacgctactttatagcctagttcagccacacgatgcgtagttccg

SEQ ID NO: 65 - DNA, 80 bp, Artificial Sequence; identical to SEQ ID NO: 61
  Synthetic DNA ligand (Afla-21-21)

SEQ ID NO: 66 - DNA, 77 bp, Artificial Sequence
  Synthetic DNA ligand (Afla-17-38)
cgctctcgtccatgtgttggcccccaaaaccgtaccatccttgcatgctaggtttttccacacgatgcgtagttccg

SEQ ID NO: 67 - DNA, 80 bp, Artificial Sequence; identical to SEQ ID NO: 52
  Synthetic DNA ligand (Afla-21-22)

SEQ ID NO: 68 - DNA, 80 bp, Artificial Sequence
  Synthetic DNA ligand (Afla-21-23)
cgctctcgtccatgtgttggagccacgaaagcgtacataactttattatctagctatgccccacacgatgcgtagttccg

SEQ ID NO: 69 - DNA, 80 bp, Artificial Sequence
  Synthetic DNA ligand (Afla-21-24)
cgctctcgtccatgtgttggtcaacgtcgtacaattcttcataattctagacgtccccggccacacgatgcgtagttccg

SEQ ID NO: 70 - DNA, 80 bp, Artificial Sequence
  Synthetic DNA ligand (Afla-21-27)
cgctctcgtccatgtgttggccatgggaaggcgtatattctttacaatctagccgacccgccacacgatgcgtagttccg

SEQ ID NO: 71 - DNA, 80 bp, Artificial Sequence
  Synthetic DNA ligand (Afla-21-28)
cgctctcgtccatgtgttggattggccatggttataaagtaaccgatggggcgatcgggaccacacgatgcgtagttccg

SEQ ID NO: 72 - DNA, 80 bp, Artificial Sequence
  Synthetic DNA ligand (Afla-21-29)
cgctctcgtccatgtgttggaaggagccgagaaaacactactttatagtgttatagtaccccacacgatgcgtagttccg

SEQ ID NO: 73 - DNA, 80 bp, Artificial Sequence
  Synthetic DNA ligand (Afla-21-30)
cgctctcgtccatgtgttggcgcattgcaggcgtatatcactttatgatctagccaggtaccacacgatgcgtagttccg

SEQ ID NO: 74 - DNA, 80 bp, Artificial Sequence; identical to SEQ ID NO: 4
  Synthetic DNA ligand (Afla-21-25)

SEQ ID NO: 75 - DNA, 80 bp, Artificial Sequence
  Synthetic DNA ligand (Afla-21-32)
cgctctcgtccatgtgttggaatttgtgcaatgtctagacgtaaagcgtatacgaattgaccacacgatgcgtagttccg

SEQ ID NO: 76 - DNA, 80 bp, Artificial Sequence
  Synthetic DNA ligand (Afla-21-34)
cgctctcgtccatgtgttggtcggctgctgttttggattataaagtaatctcttccaggcccacacgatgcgtagttccg

SEQ ID NO: 77 - DNA, 80 bp, Artificial Sequence
  Synthetic DNA ligand (Afla-21-36)
cgctctcgtccatgtgttggaaccagggggatcctttggtgggtcctgcggttctgatggccacacgatgcgtagttccg

SEQ ID NO: 78 - DNA, 80 bp, Artificial Sequence
  Synthetic DNA ligand (Afla-21-37)
cgctctcgtccatgtgttggactggcgaggggatcctctggtggtgtccttgccctttggccacacgatgcgtagttccg

SEQ ID NO: 79 - DNA, 80 bp, Artificial Sequence; identical to SEQ ID NO: 55
  Synthetic DNA ligand (Afla-21-38)

(19) United States
(12) Patent Application Publication
(10) Pub. No.: US 2012/0225494 A1
(43) Pub. Date: Sep. 6, 2012

(54) DNA LIGANDS FOR AFLATOXIN AND ZEARALENONE

(76) Inventors: Linda Chryseis Le, London (CA); Jorge Andres Cruz-Aguado, London (CA); Gregory Allen Penner, London (CA)

(21) Appl. No.: 13/391,426

(22) PCT Filed: Aug. 20, 2010
(86) PCT No.: PCT/CA2010/001292

§ 371 Date: Apr. 20, 2012

Related U.S. Application Data
(60) Provisional application No. 61/235,770, filed on Aug. 21, 2009.

(51) Int. Cl. G01N 33/566; C07H 21/04; G01N 30/02; G01N 21/64; G01N 27/62

(52) U.S. Cl. 436/501; 536/23.1

(57) ABSTRACT

The present invention relates to DNA ligands capable of binding to aflatoxin and zearalenone. The invention relates also to methods for determining the presence and concentration of aflatoxin and zearalenone in samples such as agricultural and food products, and to methods for removing or reducing the level of aflatoxin and zearalenone in samples such as agricultural and food products. The invention further relates to methods for identifying DNA ligands capable of binding to aflatoxin and zearalenone. The invention further relates to new DNA sequences.

### FIELD OF THE INVENTION

The invention relates to DNA ligands, and more particularly to DNA ligands capable of binding to aflatoxin and zearalenone. The present invention further relates to methods and use of DNA ligands capable of binding to aflatoxin and zearalenone.

### BACKGROUND OF THE INVENTION

Mycotoxins are toxins produced by fungi. Major groups of mycotoxins include aflatoxins, ochratoxin, trichothecenes (including deoxynivalenol, T2-toxin and zearalenone), fumosins and patulin. Aflatoxins are produced by certain species of Aspergillus, including Aspergillus flavus and Aspergillus parasiticus. Zearalenone is produced by certain species of Giberella.

The chemical compound aflatoxin B1 is more fully described as Cyclopenta[c]furo[3′,2′:4,5]furo[2,3-h][1]benzopyran-1,1′-dione, 2,3,6a,9a-tetrahydro-4-methoxy-, (6aR-cis)-. The molecular weight of this compound is 312.28 (g/mol).

Aflatoxin B1 has been classified by the International Agency for Research in Cancer (IARC) as a group 1 human carcinogen, and has been demonstrated to be clearly genotoxic. There are indications that the risk for primary liver cancer is higher in regions where hepatitis B is prevalent (Henry et al., (2001) In: Safety Evaluation of Certain Mycotoxins in Food. Prepared by the Fifty-sixth meeting of the Joint FAO/WHO Expert Committee on Food Additives (JECFA). FAO Food and Nutrition Paper 74. Food and Agriculture Organization of the United Nations, Rome, Italy).

European regulations stipulate a range of acceptable levels of aflatoxin B1 in animal feed from 50 ppb (parts per million) for cattle, sheep and goats, to 5 ppb for dairy animals, and young livestock. Legislation in Europe exists stating that no more than 2 ppb aflatoxin B1, and no more than 4 ppb total aflatoxin can be present in cereal products (Commission Regulation (EC) No. 1525/98) meant for human consumption. In the United States the Food and Drug Administration has set guidelines for aflatoxin at 20 ppb for food (Compliance Policy Guide (CPG) 555.400), and up to 300 ppb for mature livestock feed (CPG) 683.100). Regulatory requirements throughout the world require testing of mycotoxins such as aflatoxin B1. An alternative means of determining the content of this toxin in food and feed would be of commercial utility.

The chemical compound (4S,12E)-15, 17-Dihydroxy-4-methyl-3-oxabicyclo[12.4.0]octadeca-12, 15, 17, 19-tetraene-2,8-dione is commonly referred to as zearalenone (Zea). The molecular weight of this compound is 318.364 (g/mol). Zea is an estrogenic resorcylic acid lactone compound produced by the fungi Fusarium spp. (Diekman, M. A. and Green, M. L., (1992 J. Anim. Sci. 70:1615-1627) and as such is classified as a mycotoxin. Estrogenic effects in various animal species including infertility, vulval oedema, vaginal prolapse, and mammary hypertrophy in females, as well as feminization of males, atrophy of testes, and development of mammary glands have been documented. (Peraica et al., Bulletin of the World Health Institute, 1999, 77 (9):754-766)

Regulatory limits for zearalenone consumption have been set in Europe at 100 ppb for grains other than corn, 200 ppb for corn, 75 ppb for non-corn flour, 200 ppb for corn flour, 50 ppb for grain based foods, and 200 ppb for grain based foods targeted for infants or young children.

U.S. Pat. No. 5,475,096 (US 096), incorporated herein by reference, teaches a method for the in vitro selection of DNA or RNA molecules that are capable of binding specifically to a target molecule. U.S. Pat. No. 5,631,146, incorporated herein by reference, teaches how to use the method of US 096 to select a single stranded DNA molecule (oligonucleotide) that is capable of specifically binding to adenosine molecules.

WO 2009/086621, which is incorporated herein by reference, describes DNA ligands capable of binding the mycotoxin OTA. DNA ligands provide significant advantages over other methods for determining the concentration and detection of mycotoxins in a sample material. DNA ligands are capable of specifically binding to selected targets. A typical DNA ligand is about 20 to about 80 nucleotides in size (less than 20 and more than 80 is also possible), binds its target with nanomolar to sub-nanomolar affinity, and discriminates against closely related targets (e.g., DNA ligands will typically not bind other proteins from the same gene family).

Given the regulatory requirements of keeping the levels of aflatoxin B1 and Zea in the low pbb levels, it would be useful to provide DNA ligands capable of binding these two mycotoxins.

### SUMMARY OF THE INVENTION

In one embodiment the present invention provides for a DNA ligand that binds to aflatoxin.

In another embodiment the present invention provides for a composition comprising an effective amount of a DNA ligand that binds to aflatoxin of the present invention, and an acceptable carrier or diluent.

In another embodiment the present invention provides for a method for detecting the presence of aflatoxin in a sample characterized in that the method comprises: (a) contacting said sample to a DNA ligand capable of binding to aflatoxin to form a mixture, such that an aflatoxin/DNA ligand complex is formed in the mixture if aflatoxin is present in the sample; and (b) determining the formation of the aflatoxin/DNA ligand complex in the mixture, thereby detecting the presence of the aflatoxin in the sample.

In another embodiment the present invention provides for a method for determining the concentration of aflatoxin in a sample characterized in that said method comprises: (a) contacting said sample to a DNA ligand capable of binding aflatoxin to form a mixture, such that an aflatoxin/DNA ligand complex is formed in the mixture if aflatoxin is present in the sample; and (b) determining the concentration of the aflatoxin in the sample by measuring the amount of aflatoxin/DNA ligand complex formed in the mixture.

In a further embodiment the present invention provides for a method of removing from or reducing the level of aflatoxin in a sample characterized in that the method comprises filtering the sample through a medium having immobilized a DNA ligand that binds to aflatoxin such that aflatoxin in the sample is retained on the medium thereby removing from or reducing the level of aflatoxin in the sample.

In a further embodiment the present invention provides for a method for the identification of DNA ligands that bind to aflatoxin B1 characterized in that the method comprises: (a) contacting a random library of single stranded DNA sequences to immobilized aflatoxin B1 under conditions wherein aflatoxin B1/DNA ligand complexes are formed between the DNA ligands within the random library and the immobilized aflatoxin B1; and (b) releasing the single stranded DNA sequences from the aflatoxin/DNA complexes, wherein said released DNA sequences are the DNA ligands that bind to aflatoxin.

In one aspect of the present invention the DNA ligand that binds to aflatoxin is selected from the group consisting of: SEQ ID NO:4, SEQ ID NO:5, SEQ ID NO:9, SEQ ID NO:10, SEQ ID NO:16, SEQ ID NO:37 to SEQ ID NO:42, SEQ ID NO:45 to SEQ ID NO:47, SEQ ID NO:49, SEQ ID NO:50, SEQ ID NO:55, SEQ ID NO:57, SEQ ID NO:62, SEQ ID NO:64, SEQ ID NO:68, SEQ ID NO:73, or any functional fragments, analogues or variants thereof.

In another aspect of the present invention the aflatoxin is aflatoxin B1. In another aspect the aflatoxin is aflatoxin B2.

In yet another aspect of the present invention the DNA ligand that binds aflatoxin B1 comprises a K_{d} for aflatoxin B1 of less than 2.0E-06 M. In another aspect yet of the present invention the DNA ligand that binds to aflatoxin B2 comprises a K_{d} of less than 4.0E-07 M.

In one embodiment the present invention provides for a DNA ligand that binds to zearalenone (Zea).

In another embodiment the present invention provides for a composition comprising an effective amount of a DNA ligand that binds to Zea and an acceptable carrier or diluent.

In another embodiment the present invention provides for a method for detecting the presence of Zea in a sample characterized in that the method comprises: (a) contacting said sample to a DNA ligand capable of binding to Zea to form a mixture, such that a Zea/DNA ligand complex is formed in the mixture if Zea is present in the sample; and (b) determining whether a Zea/DNA ligand complex is formed in the mixture, thereby detecting the presence of the Zea in the sample.

In another embodiment yet the present invention provides for a method for determining the concentration of Zea in a sample characterized in that said method comprises: (a) contacting said sample to a DNA ligand capable of binding to Zea to form a mixture, such that a Zea/DNA ligand complex is formed in the mixture if Zea is present in the sample; and (b) determining the concentration of the Zea in the sample by measuring the amount of Zea/DNA ligand complex formed in the mixture.

In a further embodiment the present invention provides for a method of removing from or reducing the level of Zea in a sample characterized in that the method comprises filtering the sample through a medium having immobilized a DNA ligand that binds to Zea such that Zea in the sample is retained on the medium thereby removing from or reducing the level of Zea in the sample.

In a further embodiment the present invention provides for a method for the identification of DNA ligands that bind to Zea characterized in that the method comprises: (a) contacting a random library of single stranded DNA sequences to immobilized Zea under conditions wherein a Zea/DNA ligand complexes are formed between the DNA ligands within the random library and the immobilized Zea; and (b) releasing the single stranded DNA sequences from the Zea/DNA complexes, wherein said released DNA sequences are the DNA ligands that bind to Zea.

In one aspect of the present invention the DNA ligand that binds Zea is selected from the group consisting of: SEQ ID NO:17 to SEQ ID NO:19 or any functional fragments, analogues or variants thereof.

In another aspect of the present invention the DNA ligand that binds to Zea comprises a K_{d} for aflatoxin B1 of less than 2.6 μM.

In one embodiment the present invention provides for a method for determining the quantity of different types of aflatoxins in a sample characterized in that the method comprises: (a) contacting an aliquot of the sample with a first DNA ligand, said DNA ligand having a known effect on the fluorescence of the different types of aflatoxins; (b) contacting another aliquot of the sample with a second DNA ligand, said second DNA ligand having a known effect on the fluorescence of the different types of aflatoxins, and the effect of the first DNA ligand on the different types of aflatoxins is different from the effect of the second DNA ligand on the different types of aflatoxins; (c) using means for solving the corresponding proportions of individual aflatoxin types present in the sample based on the fluorescence effects obtained with the first DNA ligand and the second DNA ligand; (d) determining the total amount of aflatoxins in the sample based on the amount of total aflatoxin bound to the first DNA ligand, to the second DNA ligand or to both the first and second DNA ligands; and (e) determining the relative proportion of each aflatoxin based on the proportions of step (c) as a percentage of the total aflatoxin present in the sample, thereby determining the quantity of each aflatoxin present in the sample mixture.

In one embodiment the present invention provides for a DNA sequence characterized in that said DNA sequence comprises at least one nucleotide sequence selected from the group consisting of: SEQ ID NO:4 to SEQ ID NO:79.

The DNA ligands of the present invention provide significant advantages over prior art methods for the concentration and detection of aflatoxin and Zea in sample material, including:
a. DNA ligands can be chemically synthesized. As the scale of production increases the relative cost per unit of DNA ligand is reduced.
b. DNA ligands can be modified directly through the covalent attachment is of fluorophores or fluorescence quenching moieties. This means that DNA Uganda can be modified in order to directly measure the binding interaction between DNA ligand and ligand, Quantitative measurements with antibodies rely on indirect measurements such as competition analysis. This reduces sensitivity and increases cost.
c. Oligonucleotides can maintain function within higher levels of organic solvent than antibodies. This means in the case of target molecules where extraction must be performed with organic solvents, the use of DNA ligands allows more effective partitioning of the target molecule from the organic phase to a combined organic/aqueous buffer.
d. DNA ligands are more thermal stable than antibodies and can be stored for longer periods of time without a noticeable loss of function.

In general, it would be clear to one trained in the art that a DNA ligand that bound with high affinity and specificity to either aflatoxin, or zearalenone would represent an improvement over existing antibody based methods both for the concentration of aflatoxin or zearlenone prior to analysis, and for the direct, quantitative analysis of these mycotoxins' concentration in sample material.

### BRIEF DESCRIPTION OF DRAWINGS

A brief description of one or more embodiments is provided herein by way of example only and with reference to the following drawings, in which:

FIG. 1 illustrates an analysis of aflatoxin conjugation to resin.

FIG. 2 illustrates an analysis of dialysis results with aflatoxin B1 and various putative DNA ligands.

FIG. 3 illustrates the effect of DNA ligand 17-10 (SEQ ID NO:9) on the fluorescence of aflatoxin B1.

FIG. 4 A illustrates titration of DNA ligand Afla17-10 (SEQ ID NO:9) with 200 nM of aflatoxin B1.

FIG. 4 B illustrates Titration of DNA ligand Afla17-19 with 200 nM of aflatoxin B1.

FIG. 5 illustrates a competition assay between warfarin and aflatoxin B1 is for DNA ligand Afla17-19 (SEQ ID NO:10).

FIG. 6 illustrates a titration curve for DNA ligand Afla-17-6 (SEQ ID NO:16) in the presence of aflatoxin.

FIG. 7 illustrates different types of aflatoxins: B1, B2, G1 and G2. The features circled in the chemical form B1 represent the variant points among the four molecules.

FIG. 8 A illustrates fluorescence spectra of aflatoxin B1.

FIG. 8 B illustrates fluorescence spectra of aflatoxin B2.

FIG. 8 C illustrates fluorescence spectra of aflatoxin G1.

FIG. 9 illustrates a response of aflatoxin B2 to varying concentrations of the DNA ligand Afla-17-6 (SEQ ID NO:16).

FIG. 10 illustrates a response of aflatoxin B2 to varying concentrations of the DNA ligand Afla-17-2 (SEQ ID NO:4).

FIG. 11 illustrates a response of aflatoxin B1 fluoroscence to alpha-cyclodextran.

FIG. 12 illustrates a titration curve of DNA ligand Afla-17-2 (SEQ ID NO:4) with 200 nM aflatoxin B1, and 10 mM alpha cyclodextran.

FIG. 13 illustrates a putative secondary structure of the DNA ligand Afla17-2 (SEQ ID NO:4).

FIG. 14 illustrates putative secondary structures of shortened versions of DNA ligand Afla17-2 (SEQ ID NO:4). A: 17-2-2 (SEQ ID NO:46); B: Afla17-2-3 (SEQ ID NO:47); C: Afla17-2-1 (SEQ ID NO:45); D: Afla 17-2-4 (SEQ ID NO:48); E: Afla 17-2-5 (SEQ ID NO:49); F: Afla 17-2-6 (SEQ ID NO:50); and G: Afla 17-2-7 (SEQ ID NO:51).

FIG. 15 illustrates a binding curve based on enhancement of fluorescence of Zea in the presence of the DNA ligand Zea 1.4.3 (SEQ ID NO:19).

### DETAILED DESCRIPTION OF THE INVENTION

### Definitions

Unless defined otherwise, all technical and scientific terms used herein have the same meaning as commonly understood by one of ordinary skill in the art to which this invention belongs. In the case of conflict, the present specification will control. Also, unless indicated otherwise, except within the claims, the use of “or” includes “and” and vice-versa. Non-limiting terms are not to be construed as limiting unless expressly stated or the context clearly indicates otherwise (for example “including”, “having” and “comprising” typically indicate “including without limitation”). Singular forms including in the claims such as “a”, “an” and “the” include the plural reference unless expressly stated otherwise.

The details of certain embodiments of the invention are provided in the accompanying description herein. It is understood that one of ordinary skill in the art to which this invention belongs could envision other methods and materials similar or equivalent to those described herein can be used in the practice or testing of the present invention.

Other features, objects, and advantages of the invention will be apparent from the description.

The term “ligand” means a nucleic acid polymer that binds another molecule or target analyte. In a population of candidate nucleic acid polymers, a ligand is one which binds with greater affinity than that of the bulk population. In a candidate mixture there can exist more than one ligand for a given target. The ligands may differ from one another in their binding affinities for the target molecule.

The term “nucleic acid” means either DNA, RNA, single-stranded or double-stranded and any chemical modifications thereof.

Overview

The inventors have developed new methods for identifying nucleic acid ligands.

The inventors have also developed and identified novel nucleic acid ligands that specifically bind to either aflatoxin or zearalenone (Zea). It is understood by those skilled in the art that the novel nucleic acid ligands of the present invention may be involved in a variety of applications characterized by the binding of the is nucleic acid ligands of the present invention to either aflatoxin or Zea.

The present invention also relates to the discovery of new DNA sequences: SEQ ID NO:4 to SEQ ID NO:79.

Identification of DNA Ligands

In one embodiment, the novel DNA ligands of this invention may be identified using PCR-based methods for identifying DNA ligands for a specific target. A target, such as aflatoxin B1 or Zea, is immobilized on a resin in a column. A library of single stranded oligonucleotides each composed of a central region of random nucleotides flanked by sequences of known composition is applied to the immobilized target in the column. Those oligonucleotides that do not bind to the immobilized target, or bind relatively weakly are removed through repeated washes of the column with a buffer that supports DNA ligand binding. Those oligonucleotides that do bind with high affinity to the immobilized target are recovered through the addition of an excess of free molecules of the target. This elution process also provides a selection pressure for DNA ligand specificity. The recovered putative DNA ligands are PCR amplified. The amplified double stranded DNA is re-applied to a fresh column containing the immobilized target, where the process described above is repeated. This process is repeated until no further selection gains are evident in the population of oligonucleotides at which point the library is amplified, cloned and individual oligonucleotides are sequenced. Putative DNA ligands of aflatoxin B1 or Zea are synthesized based on the sequences discovered and tested for their ability to bind to the free target.

The novelty of the present invention lies in the application of this previously taught technology for the identification of DNA ligands that bind to aflatoxin and zearalenone. The small size of these targets increases the difficulty of the selection process, and it is important that the prototocol taught in this invention be followed closely to ensure reproducible results. The use of DNA ligand selection processes as known in the art may not be sufficient to achieve success. Significant improvements include but are not limited to the addition of wash steps following initial binding of the DNA library on the immobilized target, the use of increased stringency through the inclusion of more wash steps during the selection process in response to the initial levelling off of selection, and the use of free target as a means of recovering bound ligands in the elution step. Each of these innovations and the combination of the innovations as taught in this invention are of utility in achieving reliable results.

The use of double stranded selection may represent an improvement over prior art in that the single stranded amplification of DNA can often lead to artifacts, including concatemers of amplified products and rearrangements of primer sequences within the amplified products. These artifacts can overwhelm the library due to their ability to amplify more readily than the target PCR products and thus prevent effective selection for ligands from occurring.

Thus in one embodiment of the invention, the inventors were able to identify novel DNA ligands that specifically bind to aflatoxin. Using binding assays, the inventors demonstrated that the DNA ligands selected for binding to aflatoxin B1 bound with significant affinity and specificity to aflatoxin.

In one aspect the DNA ligands of the present invention that bind to aflatoxin may be selected from the group consisting of: SEQ ID NO:4, SEQ ID NO:5, SEQ ID NO:9, SEQ ID NO:10, SEQ ID NO:16, SEQ ID NO:37 to SEQ ID NO:42, SEQ ID NO:45 to SEQ ID NO:47, SEQ ID NO:49, SEQ ID NO:50, SEQ ID NO:55, SEQ ID NO:57, SEQ ID NO:62, SEQ ID NO:64, SEQ ID NO:68, SEQ ID NO:73, or any functional fragments, analogues or variants thereof.

In one aspect of the present invention the aflatoxin is aflatoxin B1. The differential effect on the fluorescence of this molecule as contrasted to other aflatoxins such as aflatoxin B2, and G1 would be useful in the creation of diagnostic assays that are capable of differentiating aflatoxin B1 from the other aflatoxins. This aspect of the invention has commercial utility in that certain regulations specify the determination of the concentration of aflatoxin B1 only rather than all aflatoxins.

In another embodiment of the present invention, the inventors used their mycotoxin identification process to identify a DNA ligand that bound to Zea. Using binding assays, the inventors demonstrated that this DNA ligand bound to Zea with sufficient affinity to enable detection of Zea at relevant concentrations of regulatory concern.

In one aspect of the present invention the DNA ligand that binds Zea is selected from the group consisting of: SEQ ID NO:17 to SEQ ID NO:19 or any functional fragments, analogues or variants thereof.

In another aspect of the present invention the DNA ligand that binds Zea comprises a K_{d} for Zea of less than 2.6 μM.

The DNA ligands of the present invention may also encompass “functionally equivalent variants” or “analogues” of the oligonucleotides. As such, this would include but not be limited to oligonucleotides with partial sequence homology, oligonucleotides having one or more specific conservative and/or non-conservative base changes which do not alter the biological or structural properties of the DNA ligand (i.e. the ability to bind to a target).

In terms of “functional analogues”, it is well understood by those skilled in the art, that inherent in the definition of a biologically functional analogue is the concept that there is a limit to the number of changes that may be made within a defined portion of a molecule and still result in a molecule with an acceptable level of equivalent biological activity, which, in this case, would include the ability to bind to aflatoxin or Zea. A plurality of distinct nucleic acid polymers with different substitutions may easily be made and used in accordance with the invention. It is also understood that certain bases are particularly important to the biological or structural properties of the DNA ligand in the mycotoxin recognition region, such bases of which may not generally be exchanged.

The DNA ligand analogues of the instant invention also encompass nucleic acid polymers that have been modified by the inclusion of non-natural nucleotides including but not limited to, 2,6-Diaminopurine-2′-deoxyriboside, 2-Aminopurine-2′-deoxyriboside, 6-Thio-2′-deoxyguanosine, 7-Deaza-2′-deoxyadenosine, 7-Deaza-2′-deoxyguanosine, 7-Deaza-8-aza-2′-deoxyadenosine, 8-Amino-2′-deoxyadenosine, 8-Amino-2′-deoxyguanosine, 8-Bromo-2′-deoxyadenosine, 8-Bromo-2′-deoxyguanosine, 8-Oxo-2′-deoxyadenosine, 8-Oxo-2′-deoxyguanosine, Etheno-2′-deoxyadenosine, N′-Methyl-2′-deoxyadenosine, 06-Methyl-2′-deoxyguanosine, 06-Phenyl-2′-deoxyinosine, 2′-Deoxypseudouridine, 2′-Deoxyuridine, 2,4-Difluorotoluoyl, 2-Thiothymidine, 4-Thio-2′-deoxyuridine, 4-Thiothymidine, 5′-Aminothymidine, 5′-Iodothymidine, 5′-O-Methylthymidine, 5,6-Dihydro-2′-deoxyuridine, 5,6-Dihydrothymidine, 5-(C2-EDTA)-2′-deoxyuridine, 5-(Carboxy)vinyl-2′-deoxyuridine, 5-Bromo-2′-deoxycytidine, 5-Bromo-2′-deoxyuridine, 5-Fluoro-2′-deoxyuridine, 5-Hydroxy-2′-deoxycytidine, 5-Hydroxy-2′-deoxyuridine, 5-Hydroxymethyl-2′-deoxyuridine, 5-lodo-2′-deoxycytidine, 5-lodo-2′-deoxyuridine, 5-Methyl-2′-deoxycytidine, 5-Propynyl-2′-deoxycytidine, 5-Propynyl-2′-deoxyuridine, 6-O-(TMP)-5-F-2′-deoxyuridine, C4-(1,2,4-Triazol-1-yl)-2′-deoxyuridine, N4-Ethyl-2′-deoxycytidine, O4-Methylthymidine, Pyrrolo-2′-deoxycytidine, and Thymidine Glycol.

The DNA ligands of the present invention may be made by any of the methods known to those of skill in the art most notably, preferably by chemical synthesis. A common method of synthesis involves the use of phosphoramidite monomers and the use of tetrazole catalysis (McBride and Caruthers, Tetrahedron Lett. (1983) 24:245-248). Synthesis of an oligonucleotide starts with the 3′ nucleotide and proceeds through the steps of deprotection, coupling, capping, and stabilization, repeated for each nucleotide added.

The novel nucleic acid ligands for aflatoxin B1 and Zea of the present invention may be involved in a variety of applications characterized by the binding of the nucleic acid ligands of the present invention to aflatoxin B1 or Zea.

Determining the Presence and/or Concentration of Aflatoxin or Zea in Samples

In one embodiment the DNA ligands of the present invention may be used for the quantitative determination of the concentration of aflatoxin and/or Zea in samples of interest. In another embodiment, the DNA ligands of the present invention may be used to determine the presence or absence of aflatoxin and/or Zea in a sample. In another embodiment, the DNA ligands of the present invention may be used to remove aflatoxin and/or Zea from a sample or reduce the level of aflatoxin and/or Zea in a sample.

It would be clear to one trained in the art that several methods exist that would enable the potential use of the DNA ligands for aflatoxin and/or Zea of the present invention for the determination of the concentration of that mycotoxin in a sample.

In one embodiment the present invention provides for methods for detecting the presence of aflatoxin and/or Zea in a sample.

In one aspect, the present invention provides for a method of detecting aflatoxin in a sample, said method comprising: (a) contacting said sample to a DNA ligand capable of binding to said aflatoxin to form a mixture, such that an aflatoxin/DNA ligand complex is formed in the mixture if said aflatoxin is present in the sample; and (b) determining the formation of the aflatoxin/DNA ligand complex in the mixture, thereby detecting the presence of the aflatoxin in the sample.

In another aspect the present invention provides for a method for detecting the presence of Zea in a sample characterized in that the method comprises: (a) contacting said sample to a DNA ligand capable of binding to Zea to form a mixture, such that a Zea/DNA ligand complex is formed in the mixture if Zea is present in the sample; and (b) determining the formation of Zea/DNA ligand complex in the mixture, thereby detecting the presence of the Zea in the sample.

In another embodiment the present invention includes methods for determining the concentration of aflatoxin and/or Zea in a sample.

In one aspect, the present invention provides for a method for determining the concentration of aflatoxin in a sample, said method comprising: (a) contacting said sample to a DNA ligand capable of binding to aflatoxin to form a mixture, such that an aflatoxin/DNA ligand complex is formed in the mixture if aflatoxin is present in the sample; and (b) determining the concentration of the aflatoxin in the sample by measuring the amount of aflatoxin/DNA ligand complex formed in the mixture.

In another aspect the method for determining the concentration of aflatoxin in a sample comprises (a) contacting said sample to a DNA ligand that binds to aflatoxin to form an aflatoxin/DNA ligand complex; (b) releasing the aflatoxin from the aflatoxin/DNA ligand complex; and (c) determining the concentration of the aflatoxin in the sample by measuring the amount of released aflatoxin.

In a further aspect, the present invention provides for a method for determining the concentration of Zea in a sample, said method comprising: (a) contacting said sample to a DNA ligand capable of binding to Zea to form a mixture, such that a Zea/DNA ligand complex is formed in the mixture if Zea is present in the sample; and (b) determining the concentration of the Zea in the sample by measuring the amount of Zea/DNA ligand complex formed in the mixture.

In a further aspect the method for determining the concentration of Zea in a sample comprises (a) contacting said sample to a DNA ligand that binds to Zea to form a Zea/DNA ligand complex; (b) releasing the Zea from the Zea/DNA ligand complex; and (c) determining the concentration of the Zea in the sample by measuring the amount of released Zea.

The immobilization of DNA ligands and their subsequent use for determination of concentration of the target molecule that the DNA ligand binds to in a sample has been achieved prior to this invention. Romig et al. (J. Chromatogr. (1999) B 731:275-284), which is incorporated herein by reference, immobilized a 5′ biotinylated DNA ligand for human L-selectin onto a streptavidin sepharose support which was then packed into a column. The target protein was eluted from the column under conditions that did not cause protein denaturation, but affected the cation support of the tertiary structure necessary for DNA ligand binding. This application resulted in a 1,500 fold purification of the target protein, with 83% recovery in a single step. Kotia et al. (Anal Chem (2000) 72:827-831), which is incorporated herein by reference, has demonstrated that immobilized DNA ligands can be used to concentrate small target molecules similar in size to mycotoxins. This group demonstrated that immobilized DNA ligands could be used to separate polyaromatic hydrocarbons such as naphthalene and benzo[a]pyrene (BaP), as well as naphthalene and benzo(ghi)perylene (BgP). Kotia et al. demonstrated that separation results were improved through the use of acetonitrile concentrations up to 60% for BaP, and methanol concentrations from 20 to 30% for BgP. The inventors of the present invention realized that the development of similar technology for the detection of mycotoxins would be useful given that the extraction of mycotoxins relies on the use of organic solvents such as methanol, ethanol or acetonitrile.

For use with immunoaffinity columns aflatoxin and/or Zea must be partitioned from organic solvents into an aqueous solvent. This step requires additional time, and results in both an increased dilution of the target molecule and implicit losses of the mycotoxin from the analysis procedure.

In WO 2009/086621 the inventors have demonstrated the use of relatively high levels of organic solvents in the affinity column without a compensatory loss in DNA ligand binding activity with a mycotoxin target. This step provides a significant advantage over antibody based methods which require substantially more dilution of the organic solvents used in extraction prior to exposure to the antibody.

As such, one embodiment of this invention includes the use of a DNA ligand in an affinity column for the determination of aflatoxin and/or Zea presence and/or concentration in a sample comprising the following steps:
(a) immobilizing a DNA ligand for aflatxoin and/or Zea to an affinity column;
(b) running an extract of the sample through the affinity column under conditions wherein an aflatoxin/DNA ligand complex and/or Zea/DNA ligand complex is formed if said aflatoxin and/or Zea is/are present in the sample;
(c) recovering the aflatxoin and/or Zea from the column with a recovering agent; and
(d) measuring the quantity of aflatxoin and/or Zea captured by the column by methods such as direct fluorescence measurement, high performance liquid chromatography and mass spectrometry of the mycotoxin target, and the use of fluorescence, or fluorescence in combination with quenchers, or fluorescence polarization, and electro-affinity analysis of the target/DNA ligand complex formation.

In one aspect of the invention the extract is an organic solvent extract of the sample. Suitable organic solvents include, but are not limited to, methanol and ethanol. In another aspect the organic extract solution may be diluted to a level where the organic solvent is tolerated by the DNA ligand (for example, 5% to 25% methanol, or 10% ethanol). The recovering agent may comprise 20% methanol without salts or 10% ethanol.

In another aspect of this invention the method for the determination of concentration of aflatoxin and/or Zea may include a washing step following the introduction of the sample to the affinity column and prior to the elution of the sample from the column.

Given that the DNA ligands of the present invention when in contact with a sample bind only to aflatoxin or Zea that may be present in the sample to form a mycotoxin/DNA ligand complex, another aspect of the present invention comprises methods for removing or reducing the level of aflatoxin or Zea in the sample. Furthermore, given that the DNA ligands of the present invention when in contact with a sample bind only to aflatoxin or Zea that may be present in the sample to form a mycotoxin/DNA ligand complex, the DNA ligands of the present invention may be used in a method for modifying the biological function of the mycotoxin, including the inhibition of the biological function of the mycotoxin. Therefore another aspect the present invention comprises methods for modifying the biological function of mycotoxins.

The presence of aflatoxin, Zea, the formation of aflatoxin/DNA complex, and/or Zea/DNA complex may be determined by any known method, including fluorescence, high performance liquid chromatography, mass spectrometry of aflatoxin or Zea, fluorescence in combination with quenchers or fluorescence polarization.

In another embodiment, the present invention includes methods for removing aflatoxin and/or Zea from a sample, or reducing the level of aflatoxin and/or Zea in a sample.

In one aspect, the present invention provides for a method for removing from or reducing the level of aflatoxin in a sample. The method comprises filtering the sample through a medium having immobilized a DNA ligand that binds to aflatoxin such that aflatoxin in the sample is retained on the medium thereby removing from aflatoxin from the sample or reducing the level of aflatoxin in the sample. In aspects of the invention the medium may be an affinity column.

In another aspect, the present invention provides for a method for removing from or reducing the level of Zea in a sample. The method comprises filtering the sample through a medium having immobilized a DNA ligand that binds to Zea such that Zea in the sample is retained on the medium thereby removing from or reducing the level of Zea in the sample.

In aspects of the invention the medium may be an affinity column.

Given that a DNA ligand based affinity column would bind only the mycotoxin present within a sample while allowing other components to flow through, an embodiment of the present invention would be the use of affinity columns consisting of DNA ligands for aflatoxin or Zea for the removal, or reduction of aflatoxin or Zea in samples, such as agricultural or food products. One embodiment of this invention would be the removal or reduction of aflatoxin in samples such as agricultural or food products through the use of an affinity column. Another embodiment of the present invention would be the removal or reduction of Zea in agricultural or food products through the use of an affinity column.

In another aspect of the present invention, the DNA ligands of the present invention may be immobilized onto lateral flow strips. The presence and/or concentration of the targets for said DNA ligands may be determined on the surface of said strips. Lateral flow strips and methods for determining the presence and/or concentration of targets on lateral flow strips are described in PCT/CA2010/001152, which is incorporated herein by reference.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1

### Identification of DNA Ligands for Aflatoxin

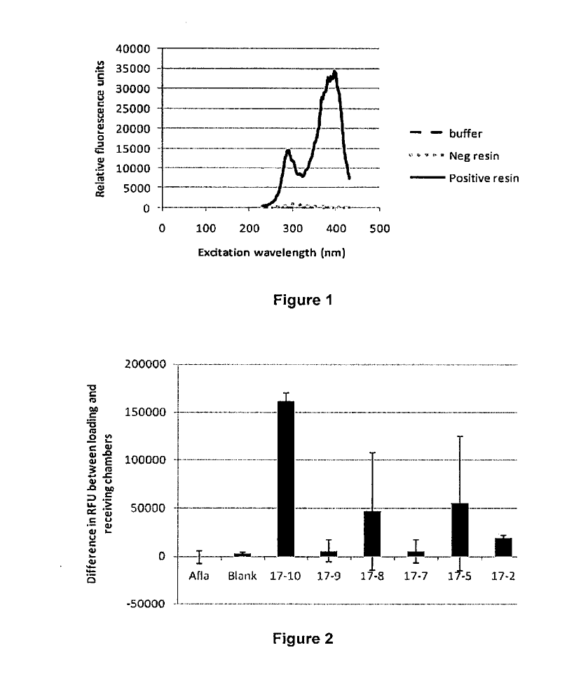

A resin CL-hydrazide PI 20391 was derivatized with aflatoxin B1 by preparing a 1 mL solution of resin. This slurry was washed once with 0.5 M acetate buffer (pH 5.5), followed by three washes with the same acetate buffer with increasing amounts of dimethyl formamide (DMF) up to a final DMF concentration of 20% (v/v). For generation of Negative Resin, 150 μL of DMF was added, while for Positive Resin, 10 mM aflatoxin B1 was added in the same 150 μL of DMF. The volume of the resin was adjusted to 2 mL with the acetate buffer. These solutions were incubated at room temperature for 3 days with rotation. They were subsequently washed ten times with 1.1 mL of Selection Buffer (10 mM Hepes, 120 mM NaCl, 5 mM KCl, 5 mM MgCl2, pH 7.0). 25 μL of the washed slurry were transferred to a UV transparent microtitre dish well, and fluorescence was read from the bottom with an excitation wavelength of 366 nm, and an emission wavelength of 430 nm. The presence of fluorescence in the resin demonstrates the presence of conjugated aflatoxin B1 (FIG. 1).

The emission spectra is shifted towards the red which one trained in the art would interpret as expected as the conjugation involves a carbonyl group and this affects the delocalization of pi electrons in aflatoxin B1.

In the present invention an initial library (SEQ ID NO:1) was created with two regions of known sequence flanking 40 nucleotides of unknown sequence. The two regions of known sequence were used as complementary sites for PCR amplification with the primers listed as SEQ ID NO:2 and SEQ ID NO:3. A quantity of this library was used that would correspond to 10¹⁵ sequences was applied to a Negative Column (120 μL Negative Resin). The library was denatured prior to application to the column by heating the DNA at a temperature of 90° C. for five minutes followed by incubation at room temperature for 30 min. The Negative Column was washed twice with 1 mL of Selection Buffer. The library was diluted into 400 μL of Selection Buffer, loaded onto the column and allowed to incubate for ten minutes at room temperature. The flow through from the negative column and one wash with Selection Buffer (400 μL) were collected and pooled (total collected 800 μL). 600 μL of this pooled fraction was then added to the Positive Column (120 μL Positive Resin). The Positive Columns were washed the desired number of times with 600 μL of Selection Buffer (Table 1).

TABLE 1
Description of selection strategy for DNA ligands for aflatoxin B1

| | Neg- | | Neg- | | | | |
|---|---|---|---|---|---|---|---|
| | ative | Positive | ative | Positive | PCR | Tem- | # of |
| Round | Binding | Binding | Elution | Elution | cycles | plate | washes |
| 1 | | | | | | | |
| 2 | | 45.0% | | | 8 | 5 ul | 2 |
| 3 | 15.0% | 5.5% | | | 16 | 5 ul | 2 |
| 4 | 10.2% | 11.1% | | | 16 | 5 ul | 2 |
| 5 | 6.3% | 5.9% | | | 16 | 5 ul | 2 |
| 6 | 8.6% | 7.5% | | | 16 | 5 ul | 2 |
| 7 | 0.0% | 3.8% | | | 14 | 5 ul | 2 |
| 8 | 0.0% | 12.8% | 1.2% | 1.8% | 15 | 5 ul | 2 |
| 9 | 2.5% | 16.3% | 1.6% | 4.7% | 13 | 5 ul | 2 |
| 10 | 10.9% | 16.1% | 1.0% | 4.9% | 14 | 5 ul | 2 |
| 11 | 8.8% | 15.0% | 0.8% | 6.5% | 12 | 5 ul | 2 |
| 12 | 9.2% | 16.9% | 0.8% | 9.0% | 10 | 5 ul | 2 |
| 13 | 14.6% | 23.3% | 1.4% | 14.4% | 10 | 5 ul | 2 |
| 14 | 11.8% | 22.6% | 0.9% | 13.4% | 14 | 2 ul | 2 |
| 15 | 9.5% | 23.0% | 0.8% | 13.2% | 12 | 2 ul | 4 |
| 16 | 4.6% | 20.7% | 0.3% | 11.2% | 14 | 2 ul | 4 |
| 17 | 6.8% | 25.2% | 0.3% | 12.1% | 13 | 2 ul | 4 |
| 18 | 15.8% | 28.2% | 0.3% | 14.0% | 16 | 2 ul | 4 |
| 19 | 13.2% | 24.5% | 0.2% | 7.0% | 16 | 2 ul | 10 |
| 20 | 7.9% | 23.8% | 0.3% | 8.1% | 16 | 5 ul | 10 |
| 21 | 8.2% | 24.2% | 0.2% | 5.9% | 16 | 5 ul | 10 |

Nucleic acid polymers that remained bound to the Positive Column following washes were eluted with two consecutive additions of 200 μL of 500 μM aflatoxin B1 in Selection Buffer. Each elution was incubated for ten minutes prior to collection. The two elutions were combined and the DNA amplified using a polymerase chain reaction (PCR) strategy. The inventors had intended for the strategy to result in 1.0 asymmetric amplification of the DNA and as such used 1 μM sense primer (with a fluorescent label (Hex)) in combination with 25 nM unlabeled reverse primer. A test PCR reaction was used to determine the appropriate number of rounds of selection necessary to obtain adequate amplification of the library. Once the appropriate number of cycles was ascertained, a total of twenty four, 100 μL reactions were performed using the same conditions. Each reaction contained 5 μL of template DNA, unless the number of PCR cycles required was less than ten, in which case the template amount was reduced to 2 μL/reaction. Each reaction contained 1×PCR buffer (New England BioLabs), six units of Taq polymerase, 200 nM each dNTP, and the concentrations of primers noted above. Following PCR amplification unincorporated primers were removed through the use of Qiagen Quick Elute kits. The amount of single stranded DNA amplified was estimated through quantification of electrophoretic bands in agarose gels. Amplified DNA was pooled and diluted to a total of 420 μL prior to inclusion in the subsequent cycle of selection.

After the experiment was concluded the inventors noticed that no more sense strand was amplified than antisense strand. The inventors created a probe for the sense strand using a biotinylated version of the reverse primer. The amplified products from the final selection round were combined with this biotinylated probe without a prior denaturation step. In this way, only those sense strands that were not associated with an antisense strand would have the capacity to bind to the biotinylated probe. If all of the library consisted of sense strands then one would expect all the fluorescence present in the sample prior to this test to be lost from the as they form complements with the biotinylated reverse primer, and the biotinylated probe binds to immobilized streptavidin. This was not the case, almost all the fluorescence was recovered from the elution product, indicating that the majority of the DNA was in fact double stranded.

After selection cycle 15, a counter selection step was introduced whereby following the loading of the Positive Column with the selected DNA library, a 500 uM concentration of warfarin in Selection Buffer was added to the column. This was washed three times followed by an additional three washes prior to aflatoxin B1 elution.

In the last selection cycle PCR was performed using unlabeled versions of SEQ ID NO:2 and SEQ ID NO:3. The PCR product was then ligated into pGEM-T vectors and cloned into E. coli to facilitate clone sequencing.

It would be clear to one trained in the art that several inventive steps have been enabled by this process. When the number of PCR cycles necessary to regenerate the starting amount of DNA appeared to definitely plateau and was no longer exhibiting a decrease, an increase in the number of washes was imposed. The imposed increase in the number of washes allowed for a greater level of optimization between the stringency of selection and the probability of eliminating the best binding sequences from the library. This balance may be crucial to the success of the selection process.

The nature of the immobilization of aflatoxin B1, followed by the use of this immobilized molecule to select for DNA ligands is novel and had not previously been demonstrated.

Binding Assays

A total of 15 clones from 17 rounds of selection were sequenced (SEQ ID NOs.:4-16).

TABLE 2
Comparison of random regions from round
17 selection for aflatoxin B1

| | | SEQ |
|---|---|---|
| | | ID |
| Clone | DNA SEQUENCE | NO. |
| 17-3 | GCAGGATTGAGTATAAAGTACTAAATCTATCCGACCTGTG | 15 |
| 17-11 | GCACGTGTTGTCTCTCTGTGTCTCGTGCCCTTCGCTAGGC | 12 |
| 17-12 | GAGTAGCTATACAAACGTATCACTTTATGCTAGTTTGTCG | 13 |
| 17-15 | CAGGGAGGAGGAATTATAAAGTAATTCCTAATGTGCAGTA | 14 |
| 17-18 | GAACCCCATAATTCACTGTATAAAGTACTGTGAATCACCG | 11 |
| 17-2 | GCACGTGTTGTCTCTCTGTGTCTCGTGCCCTTCGCTAGGC | 4 |
| 17-5 | CTGCGTCCCTTCGTCGTCTCCCTGTGCTCGGAAGGGATTG | 5 |
| 17-7 | GCAGCTAAAATTATAAAGTAATTCTATGCTGGTTTAGGGG | 5 |
| 17-8 | CAATGTCGGCATGGCCATCTATAAAGTAGATGGTGTGCCC | 7 |
| 17-9 | GCGGATAGCAGGTAACGGATCCGCTATCCTATCGCCACAG | 8 |
| 17-10 | CGTGACGCCCGTCGTATGTACTTTATACCTAGACGTGCGC | 9 |
| 17-19 | CGTGACGCCCGTCGTATGTACTTTATACCTAGACGTGCGA | 10 |
| 17-6 | GGGCGCCGTATCGTACTTTATACGCTAGGCCTTCGTTTGC | 16 |

Several consensus motifs are evident within the random region of the sequences, as shown in Table 2. Six of these clones were tested for their potential binding affinity with aflatoxin B1 through dialysis and through measurement of aflatoxin B1 fluorescence in the presence of each putative DNA ligand. Microequilibrium dialyzers (Harvard Apparatus) were loaded with selection buffer in the receiving chamber and Selection Buffer containing 200 nM aflatoxin B1 and 5 μM of a specific DNA ligand in the loading chamber. Dialysis was allowed to proceed for 48 h at room temperature. Two replicates were performed for each of the DNA ligands tested. The affinity of DNA ligands to aflatoxin B1 was estimated by measuring the intrinsic fluorescence of aflatoxin in the loading (Fl) and receiving (Fr) chambers. The fraction of bound aflatoxin B1 (f) was then determined as:

$$f = \frac{{F}_{l} - {F}_{r}}{{F}_{l}} \qquad ( 1 )$$

The dissociation constant (K_{d}) was estimated as follows;

$$Kd = \frac{[ {A}_{0} ]}{f} - [ {A}_{0} ] \qquad ( 2 )$$

where [A₀] is the total concentration of the DNA ligand.

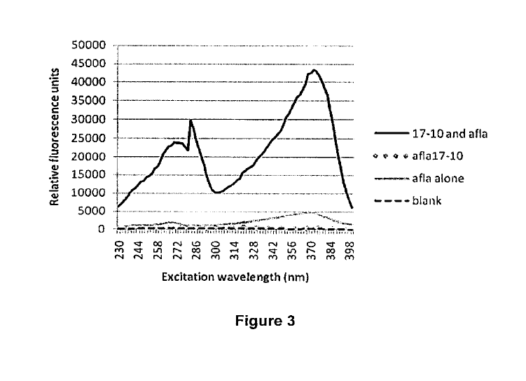

FIG. 2 shows that using the dialysis process the DNA ligand afla 17-10 (SEQ ID NO:9) demonstrated a significant binding effect with this test, while the remaining DNA ligands did not. For DNA ligands 17-8 (SEQ ID NO:7) and 17.5 (SEQ ID NO:5) the standard deviation of the estimate was high, this may have been due to an artifact disturbing the dialysis process. Using the formulae described above the K_{d} of the DNA ligand 17-10 (SEQ ID NO:9) for aflatoxin B1 was estimated at approximately 6 μM based on these dialysis results.

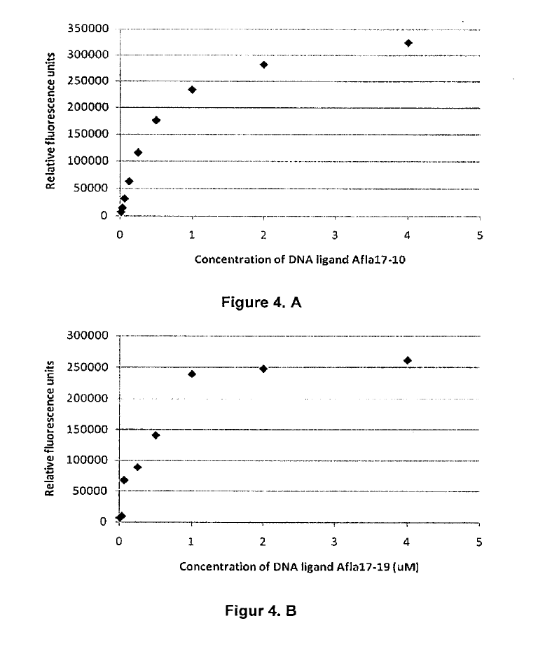

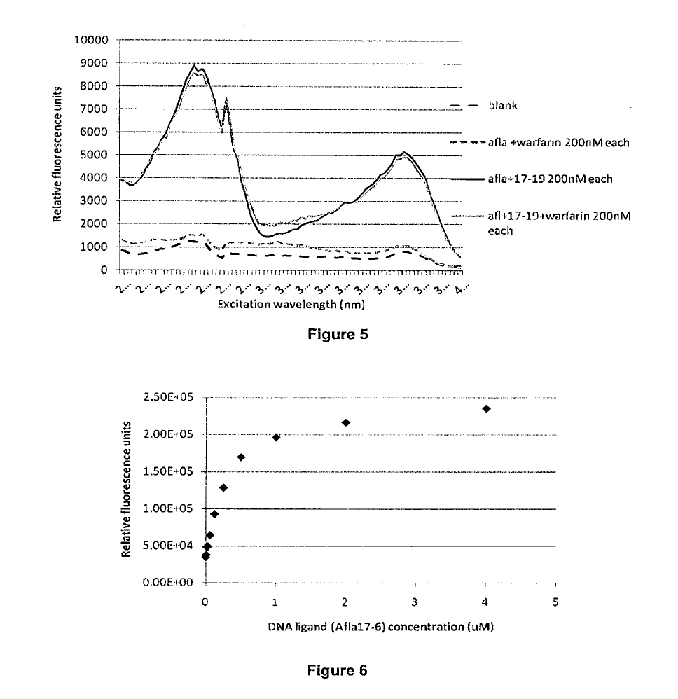

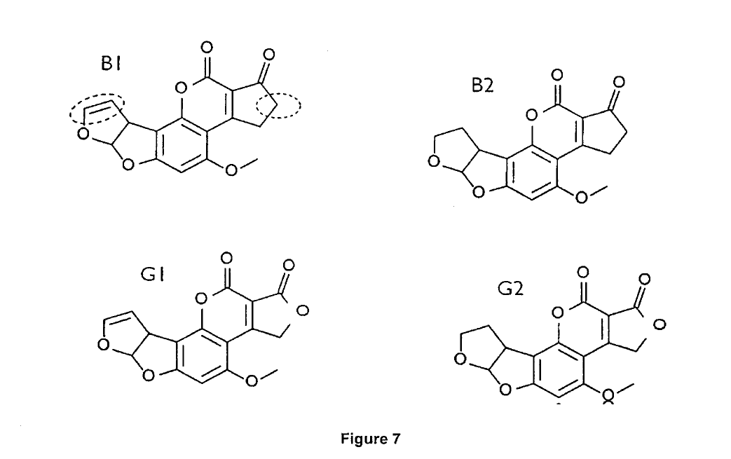

It is possible that the binding of aflatoxin B1 to a DNA ligand could result in an enhancement of the fluorescence of the aflatoxin B1 molecule, should the binding result in the removal of water molecules from the mycotoxin. This was tested by combining 100 μM concentration of aflatoxin B1 with of each of the DNA ligands, Afla17-2 (SEQ ID NO:4), Afla17-5 (SEQ ID NO:5), Afla17-7 (SEQ ID NO:6), Afla17-8 (SEQ ID NO:7), Afla17-9 (SEQ ID NO:8) and Afla17-10 (SEQ ID NO:9) and comparing the excitation spectrum to 100 μM aflatoxin B1 alone. The excitation spectrum was measured from 230 nm to 400 nm with emission at 430 nm. As illustrated in FIG. 3 aflatoxin B1 only exhibited enhanced fluorescence in the presence of the DNA ligand Afla-17-10 (SEQ ID NO:9). Given that the sequences for Afla-17-10 (SEQ ID NO:9) and Afla-17-19 (SEQ ID NO:10) were identical except for a single nucleotide, a titration of both DNA ligands was tested for binding affinity to aflatoxin B1 (200 nM) using this fluorescence enhancement test. (FIGS. 4, A and B). The K_{d} was determined based on these curves as 420 nM for Afla-17-10 (SEQ ID NO:9), and 378 nM for Afla-17-19 (SEQ ID NO:10).

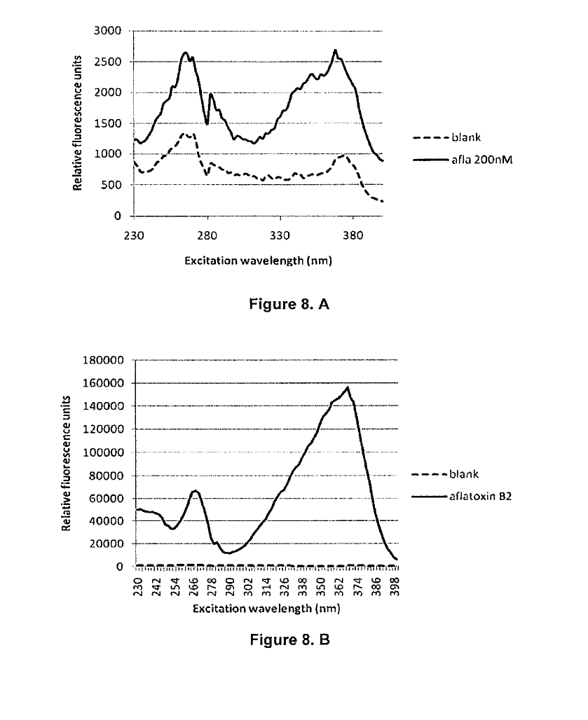

The specificity of the binding of the DNA ligand Afla-17-19 (SEQ ID NO:10) was tested with a competition assay with warfarin. Warfarin is another polycyclic aromatic molecule that also contains a lactone ring. Aflatoxin and the DNA ligand were combined at concentrations of 200 nM each, and the characteristic fluorescence enhancement exhibited by the binding of this DNA ligand to the mycotoxin was observed at a peak of 368 nm. The addition of either 200 nM or even 2 uM warfarin to this complex did not decrease the fluorescence enhancement exhibited by aflatoxin B1, thus demonstrating that aflatoxin B1 was not displaced from the DNA ligand by warfarin (FIG. 5).

### Example 2

### Further Characterization of DNA Ligand Afla-17-6 (SEQ ID NO:16) Across Various Types of Aflatoxin

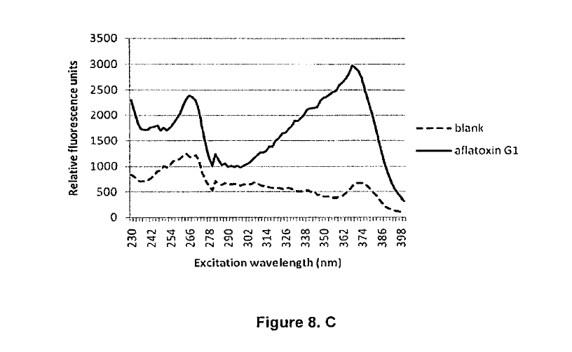

The DNA ligand Afla17-6 (SEQ ID NO:16) was shown to exhibit an even stronger binding affinity to aflatoxin B1 than the DNA ligand Afla17-10 (SEQ ID NO:9), as evidenced by the titration binding curve presented in FIG. 6. This curve results in an estimated binding affinity (K_{d}) of 220 nM.

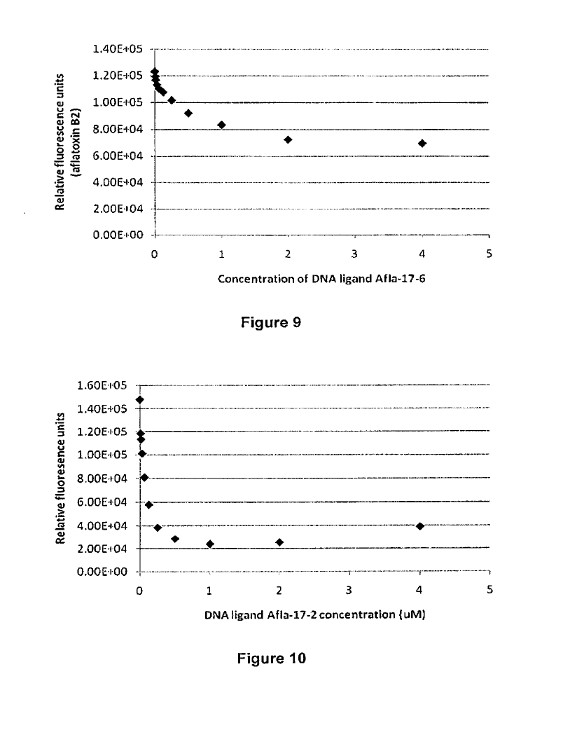

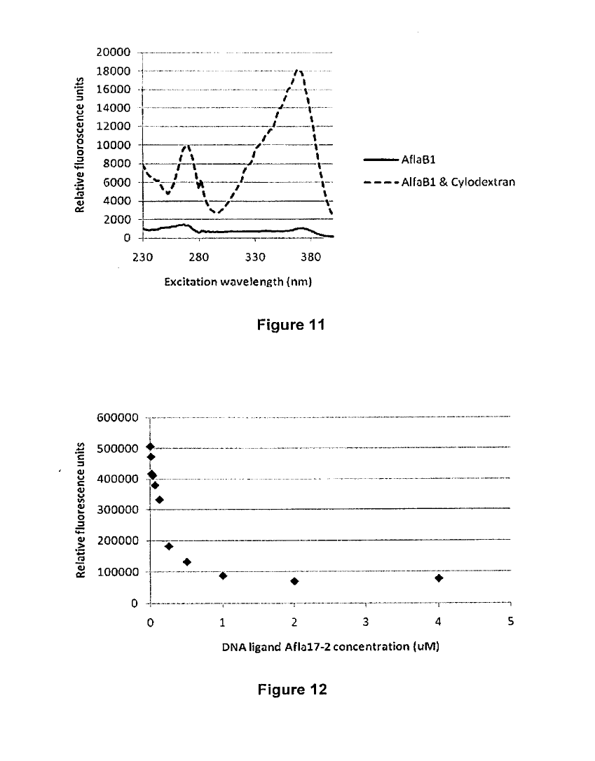

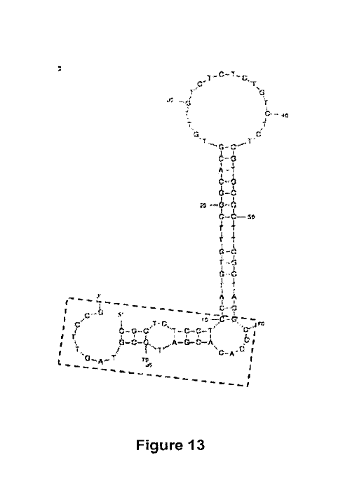

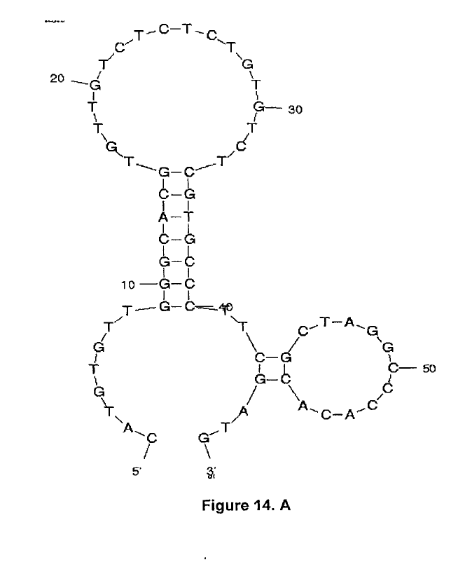

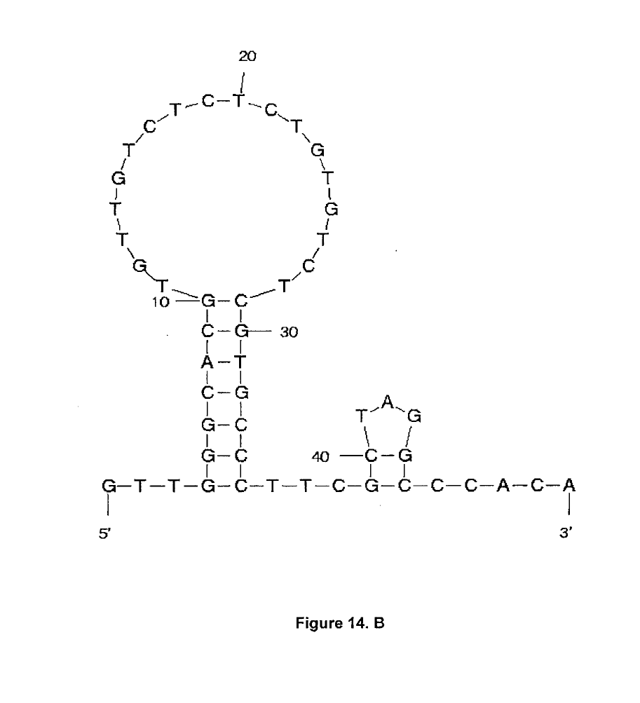

There are several different types of aflatoxins. The structures of aflatoxin B1, aflatoxin B2, aflatoxin G1 and aflatoxin G2 are shown in FIG. 7. The intrinsic fluorescence of these molecules differs significantly as shown in FIG. 8. The combination of the DNA ligand Afla-17-6 (SEQ ID NO:16) with aflatoxin B2 results in a decrease in fluorescence (FIG. 9). This binding curve indicates a binding is coefficient (K_{D}) of approximately 400 nM, or about twice as low an affinity to aflatoxin B1. The binding of this DNA ligand is enhanced at very high concentrations with aflatoxin G1. The binding curve in this case did not plateau at micromolar levels and as such it was not possible to define a K_{D} for this combination.

### Example 3

### Characterization of DNA Ligand Afla-17-2 (SEQ ID NO:4)

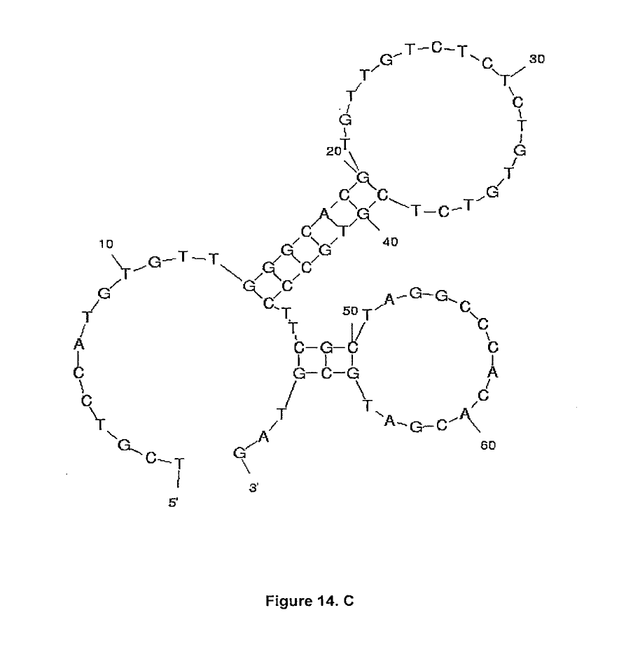

SEQ ID NO:4 did not appear to enhance the fluorescence of aflatoxin B1. SEQ ID NO:4 does, however, decrease the fluorescence of aflatoxin B2, as shown in FIG. 10. This binding curve results in an estimated binding affinity (K_{d}) of 40 nM for aflatoxin B2, much lower than that seen with the DNA ligands that did have an effect on aflatoxin B1 fluorescence.

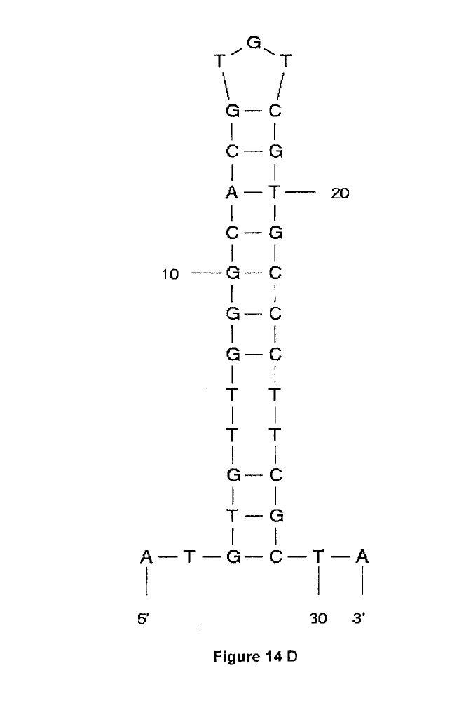

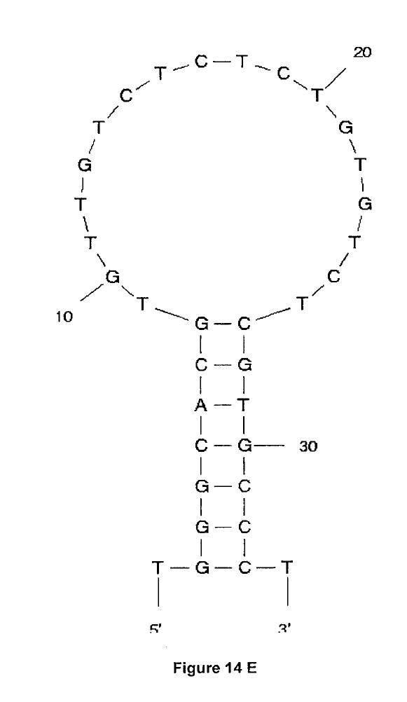

Alpha-cyclodextran is a circular glucose polymer that weakly binds aflatoxin B1. As a result of binding within the cyclodextran ring alfatoxin B1 fluorescence is enhanced. The inventors combined a 200 nM concentration of aflatoxin B1 with a 10 mM concentration of alpha-cyclodextran and determined the increased in fluorescence (FIG. 11). This fluorescence enhancement enabled the inventors to determine the binding affinity of the Afla.17-2 DNA ligand (SEQ ID NO:4) on aflatoxin B1, by measuring the competition between this DNA ligand at various concentrations and the cyclodextran at 10 mM, aflatoxin at 200 nM (FIG. 12). This titration curve results in an estimated K_{d} for binding aflatoxin B1 of 60 nM, a value similar to the estimated K_{d} for the same DNA ligand for aflatoxin B2, and much lower than other DNA ligands identified.

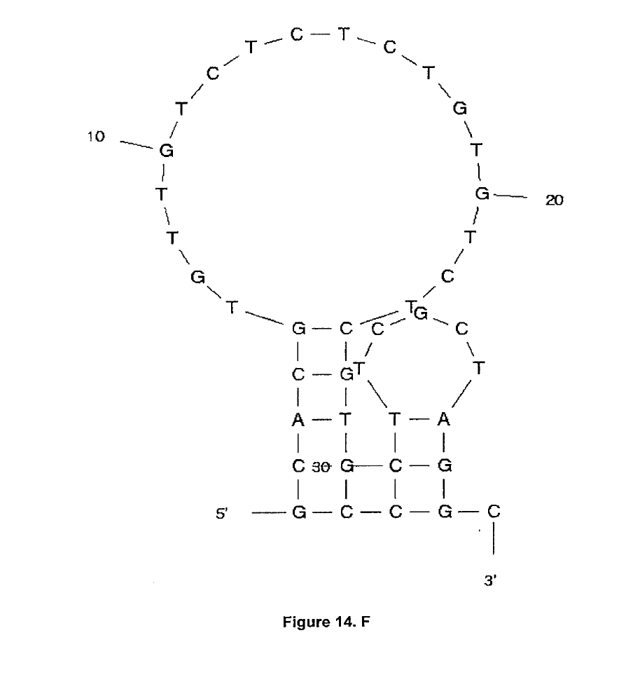

FIG. 13 provides the secondary structure of the DNA ligand Afla17-2 (SEQ ID NO:4) obtained using the Mfold web server for nucleic acid folding and hybridization prediction (http://mfold.bioinfo.rpi.edu/cgi-bin/dna-form1.cgi; M. Zuker. Nucleic Acids Res. 31 (13), 3406-15, (2003), which is incorporated herein by reference). The boxed region at the bottom of FIG. 13 contains the primer recognition sequences. The random sequence region of this DNA ligand appears to form a very simple stem and loop structure. Various shorter versions of SEQ ID NO:4 were tested to determine what portions of the structure of SEQ ID NO:4 were necessary for the maintenance of binding activity (Table 3).

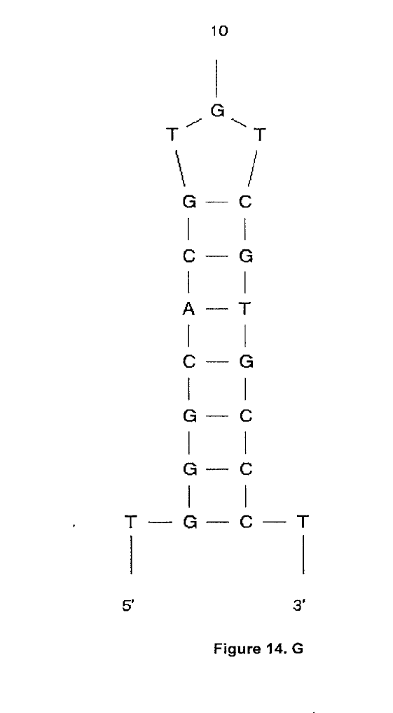

The structures represented by these shorter sequences (SEQ ID NOs.:45-51) are provided in FIG. 14. It is very clear that there is a strong correlation between the presence of the open loop attached to a stem and the ability to bind to either form of aflatoxin. The two DNA ligands that do not exhibit an open loop, Afla17-2-4 (SEQ ID NO:48, Figure D) and Afla17-2-7 (SEQ ID NO:51, Figure G) both do not exhibit binding to aflatoxin. Of these shorter versions of Afla17-2 (SEQ ID NO:4) the ligand with the strongest affinity to aflatoxin B1 was Afla17-2-3 (SEQ ID NO:47, FIG. 14.B) at a K_{d} of 75 nM, a similar value to that of the mother ligand. This would indicate that the trimming of the stem regions removed in the case of this sequence were of limited importance in binding to aflatoxin.

Table 3 provides for the binding affinity of DNA ligands for aflatoxin B1 and aflatoxin B2 as determined by the titration methods provided herein.

TABLE 3
Binding affinity of DNA Ligands
for Aflatoxin B1 and Aflatoxin B2

| DNA Ligand | SEQ ID NO | K_{d} for afla B1 | K_{d} for Afla B2 |
|---|---|---|---|
| afla 17-2 | 4 | 6.00E−08 | 4.00E−08 |
| afla 17-2-1 | 45 | 2.50E−07 | 7.00E−08 |
| afla 17-2-2 | 46 | 2.00E−07 | 3.00E−08 |
| afla 17-2-3 | 47 | 1.20E−08 | 3.00E−08 |
| afla 17-2-4 | 48 | no binding observed | no binding observed |
| afla 17-2-5 | 49 | 1.00E−07 | 5.00E−08 |
| afla 17-2-6 | 50 | 3.00E−07 | 5.00E−08 |
| afla 17-2-7 | 51 | | no binding observed |
| afla 17-5 | 5 | | 2.40E−07 |
| afla 17-6 | 16 | 2.20E−07 | 4.00E−07 |
| afla 17-6-1 | 37 | 6.18E−07 | |
| afla 17-6-2 | 38 | 2.79E−07 | |
| afla 17-6-2-1 | 42 | 3.00E−07 | |
| afla 17-6-3 | 39 | 3.09E−07 | |
| afla 17-6-4 | 40 | 3.83E−07 | |
| afla 17-6-5 | 41 | 3.57E−07 | |
| afla 17-7 | 6 | no observable binding | |
| afla 17-8 | 7 | no observable binding | |
| afla 17-10 | 9 | 4.20E−07 | |
| afla 17-12 | 13 | 6.50E−07 | |
| afla 17-19 | 10 | 3.78E−07 | |
| afla 21-5 | 55 | 1.50E−06 | |
| afla 21-8 | 57 | 2.00E−06 | |
| afla 21-14 | 62 | 4.50E−07 | |
| afla 21-16 | 64 | 7.50E−07 | |
| afla 21-23 | 68 | 3.90E−07 | |
| afla 21-30 | 73 | 8.00E−07 | |

### Example 4

### Identification of DNA Ligand for Zearalenone

The present inventors had previously enabled a strategy for the identification of DNA ligands for mycotoxins with the use of ochratoxin A as a representative mycotoxin (Cruz-Aguado and Penner, (2008), J. Agric. Food Chem., 56(22), 10456-10461, incorporated herein by reference). The oligonucleotides selected for binding to ochratoxin A (OTA) were also tested for their potential to bind to zearalenone. One such oligonucleotide, designated OTA-1.4 (SEQ ID NO:17) in the manuscript referenced above exhibited little or no binding to OTA, but did exhibit binding to zearalenone.

The binding affinity of the oligonucleotide OTA1.4 (Seq ID NO:17) for zearalenone (Zea) was demonstrated through the use of equilibrium dialysis. A buffer (Buffer H) composed of 10 mM HEPES pH 7.0, 120 mM NaCl, 5 mM KCl, and 5 mM MgCl₂ was loaded into receiving chamber of disposable equilibrium dialysers (Harvard Aparatus) and an equal volume of a solution of 2 uM Zea and 50 uM oligonucleotide in Buffer H was added to the loading chamber. Dialysis was allowed to reach equilibrium over a 48 hour period at room temperature. Binding was assessed by measuring the intrinsic fluorescence of Zea in both chambers. To measure Zea fluorescence, 65 uL aliquots from each chamber were combined with 200 uL of 60% MeOH. This was added in order to dissociate the bound Zea from the DNA ligand. Fluorescence was measured with an excitation wavelength of 316 nm, and an emission wavelength of 440 nm. The fraction of bound Zea (f) and the K_{d} was determined as described for aflatoxin B1 in Example 1 herein.

Seq ID NO:17 demonstrated a binding percentage of 95% and K_{d} of 2.6 μM under these conditions.

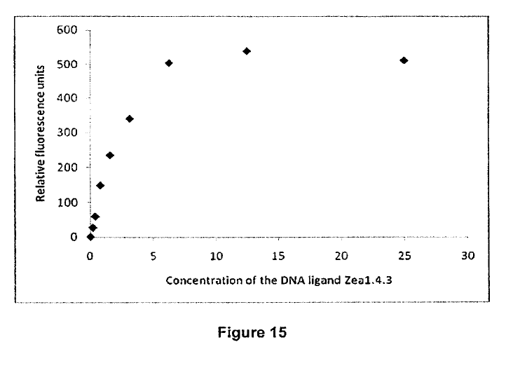

Several shortened versions of this sequence (SEQ ID NO:18-20, Zea1.4.2 to Z1.4.4) were tested for retention of Zea binding capacity. It had been observed that the binding of Zea by SEQ ID NO:17 resulted in an increase in the intrinsic fluorescence of Zea. This increase is due to two factors, the actual binding of the Zea molecule to DNA polymer, and the subsequent aggregation of bound DNA polymer to each other. As the second factor was not related to binding per se, but rather was a direct linear function of the concentration of the DNA polymer in solution, this slope was subtracted after binding had reached saturation. FIG. 15 provides the data for one DNA polymer tested, SEQ ID NO:19 (Zea1.4.3). The concentration of Zea was held constant at 500 nM and the concentration of the DNA polymer was varied. FIG. 15 provides the binding curve following the subtraction of the aggregation factor from the total fluorescence measured. Zea-1.4.2 and Zea1.4.3 (SEQ ID NOs.:18 and 19) exhibited binding to Zea while Z1.4.4 (SEQ ID NO:20) did not. The K_{d} for Z1.4.3 (Sequence ID NO:19) was calculated based on this binding curve as 2.2 uM±0.4 μM.

1. A DNA ligand that binds to aflatoxin.

2. The DNA ligand of claim 1, characterized in that said DNA ligand comprises a nucleotide sequence selected from the group consisting of: SEQ ID NO:4, SEQ ID NO:5, SEQ ID NO:9, SEQ ID NO:10, SEQ ID NO:16, SEQ ID NO:37 to SEQ ID NO:42, SEQ ID NO:45 to SEQ ID NO:47, SEQ ID NO:49, SEQ ID NO:50, SEQ ID NO:55, SEQ ID NO:57, SEQ ID NO:62, SEQ ID NO:64, SEQ ID NO:68, SEQ ID NO:73, or any functional fragments, analogues or variants thereof.

3. The DNA ligand of claim 1 characterized in that said aflatoxin is aflatoxin B1.

4. The DNA ligand of claim 3 characterized in that said DNA ligand comprises a nucleotide sequence selected from the group consisting of: SEQ ID NO:4, SEQ ID NO:9, SEQ ID NO:10, SEQ ID NO:16, SEQ ID NO:37 to SEQ ID NO:42, SEQ ID NO:45 to SEQ ID NO:47, SEQ ID NO:49, SEQ ID NO:50, SEQ ID NO:55, SEQ ID NO:57, SEQ ID NO:62, SEQ ID NO:64, SEQ ID NO:68, SEQ ID NO:73, or any functional fragments, analogues or variants thereof.

5. The DNA ligand of claim 3, characterized in that said DNA ligand comprises a K_{D} for aflatoxin B1 of less than 2.00E-06 M.

6. The DNA ligand of claim 1 characterized in that said aflatoxin is aflatoxin B2.

7. The DNA ligand of claim 6 characterized in that said DNA ligand comprises a nucleotide sequence selected from the group consisting of SEQ ID NO:4, SEQ ID NO:5, SEQ ID NO:16, SEQ ID NO:45 to SEQ ID NO:47, SEQ ID NO:49, SEQ ID NO:50, or any functional fragments, analogues or variants thereof.

8. The DNA ligand of claim 6 characterized in that said DNA ligand comprises a K_{D} for aflatoxin B2 of less than 4.00E-07 M.

9-21. (canceled)

22. A method for determining the concentration of aflatoxin in a sample characterized in that said method comprises:
(a) contacting said sample to a DNA ligand of claim 1 to form a mixture, such that an aflatoxin/DNA ligand complex is formed in the mixture if aflatoxin is present in the sample; and
(b) determining the concentration of the aflatoxin in the sample by measuring the amount of aflatoxin/DNA ligand complex formed in the mixture.

23. The method of claim 22 characterized in that the amount of aflatoxin/DNA ligand complex in the mixture is measured by fluorescence, high performance liquid chromatography, mass spectrometry of the aflatoxin, or by fluorescence in combination with quenchers or fluorescence polarization.

24. The method of claim 22 characterized in that the sample is an agricultural product or a food product.

25. The method of claim 22 characterized in that said DNA ligand comprises a nucleotide sequence selected from the group consisting of: SEQ ID NO:4, SEQ ID NO:9, SEQ ID NO:10, SEQ ID NO:16, SEQ ID NO:37 to SEQ ID NO:42, SEQ ID NO:45 to SEQ ID NO:47, SEQ ID NO:49, SEQ ID NO:50, SEQ ID NO:55, SEQ ID NO:57, SEQ ID NO:62, SEQ ID NO:64, SEQ ID NO:68, SEQ ID NO:73, or any functional fragments, analogues or variants thereof.

26. The method of claim 22 characterized in that said DNA ligand is immobilized in an affinity column or onto a lateral flow strip.

27. The method of claim 22 characterized in that said method comprises after step (a) releasing the aflatoxin from the aflatoxin/DNA ligand complex and determining the concentration of the aflatoxin in the sample by measuring the amount of released aflatoxin.

28-32. (canceled)

33. A DNA ligand that binds to zearalenone (Zea), characterized in that said DNA ligand comprises a nucleotide sequence selected from: SEQ ID NO:17 to SEQ ID NO:19 or any functional fragments, analogues or variants thereof.

34-56. (canceled)

57. A method of determining the concentration of aflatoxin in a sample comprising: (a) immobilizing a DNA ligand of claim 1 on a resin, (b) passing through the resin with the bound DNA ligand a sample suspected of containing aflatoxins, (c) washing the resin with a solution free of aflatoxins, and (d) releasing any aflatoxin bound to the resin by adding a solvent that removes the ability for the DNA ligand to bind to the aflatoxin thereby creating elution fractions, whereby the concentration of the aflatoxins present in the elution fractions is determined by direct measurement of the fluorescence of the aflatoxins.

58. The method of claim 57 characterized in that the concentration of aflatoxins in the eluted fractions is determined through high performance liquid chromatography (HPLC).

59. The DNA ligand of claim 3, characterized in that said DNA ligand is bound to an affinity column or onto a lateral flow strip.

60. The DNA ligand of claim 33 characterized in that said DNA ligand is bound to an affinity column or onto a lateral flow strip.
